(19) United States
(12) Patent Application Publication
(10) Pub. No.: US 2008/0220532 A1
(43) Pub. Date: Sep. 11, 2008

(54) Separation of Charged Solutes by Electrostatic Repulsion-Hydrophilic Interaction Chromatography

(75) Inventor: Andrew J. Alpert, Ellicott City, MD (US)

Correspondence Address: BANNER & WITCOFF, LTD., 1100 13th STREET, N.W., SUITE 1200, WASHINGTON, DC, 20005-4051, US

(73) Assignee: PolyLC Inc., Columbia, MD (US)

(21) Appl. No.: 11/683,812

(22) Filed: Mar. 8, 2007

(51) Int. Cl. G01N 30/02

(52) U.S. Cl. 436/86; 436/94

(57) ABSTRACT

In one aspect, a method of performing electrostatic repulsion-hydrophilic interaction chromatography on a protein, peptide, or amino acid includes providing a column having an anion-exchange material at a pH of less than about 4, and eluting the compound using a mobile phase comprising an amount of organic solvent sufficient to substantially balance the electrostatic repulsion of the stationary phase with hydrophilic interaction. In another aspect, a method of performing electrostatic repulsion-hydrophilic interaction chromatography on a nucleic acid or nucleotide comprises providing a column having a cation-exchange material at a pH of less than about 3.4, and eluting the compound using a mobile phase comprising organic solvent sufficient to substantially balance the electrostatic repulsion of the stationary phase with hydrophilic interaction.

### FIELD OF THE INVENTION

The invention relates to the area of chromatography methods and, more particularly, to separation of charged solutes by hydrophilic interaction chromatography.

### BACKGROUND OF THE INVENTION

Solutes in a mixture can differ greatly in their properties. In reversed-phase chromatography (RPC), this concerns differences in polarity. For ion-exchange chromatography, this concerns differences in charge. An elution gradient of some sort is generally used to insure that all solutes in a mixture elute in the same time frame.

The term Hydrophilic Interaction Chromatography (HILIC) was coined in 1990 [1] to describe normal-phase chromatography with mobile phases that, typically, are 10-40% aqueous. A sufficiently polar stationary phase material is more polar than this mobile phase and will retain polar solutes. A model for the retention mechanism postulates partitioning between the dynamic mobile phase and a slow-moving layer of water with which the polar stationary phase is hydrated. The more polar a solute, the more it associates with this stagnant aqueous phase and the later it elutes, a normal phase direction. HILIC was used for carbohydrate analysis as early as 1975 [2,3]. The mechanism of separation was recognized as early as 1967, in the case of Sephadex eluted with a predominantly organic mobile phase [4]. HILIC is useful for analysis of polar solutes in general as reversed-phase chromatography (RPC) is for nonpolar solutes. Since 1990, HILIC has been applied to a wide variety of peptides [5-10], complex carbohydrates [11], and some proteins [12-15], and is increasingly being applied to small polar solutes such as pharmaceuticals [16-17], saponins [18], urea [19], aminoglycoside antibiotics [20], glucosinolates [21], sugars and glycans [22-24], folic acid and its metabolites [25], nicotine and its metabolites [26], and glycoalkaloids [27]. Yoshida has written a series of papers examining the variables involved in HILIC of peptides [28-30]. Hemstrom and Irgum have published an ambitious paper that reviews the entire field and also attempts to ascertain the extent to which partitioning or adsorption account for the separation mechanism [31]. Gradients for elution involve increasing the polarity of the mobile phase, as in regular normal phase chromatography. Typically this involves decreasing concentrations of organic solvent, although increasing salt concentrations can be used too. Hydrophilic interaction can be superimposed as a mixed-mode on an ion-exchange column by running an increasing salt gradient in a mobile phase containing 60-70% organic solvent. These conditions work well to resolve histone variants on a weak cation-exchange column [32-37], and have also been used for chromatography of peptides on a strong cation-exchange column in an extensive series of papers from Robert Hodges' group [38-40].

Hydrophilic interactions usually determine the elution profile using HILIC. However, under certain conditions, electrostatic effects can also influence and sometimes complicate the elution of charged solutes. For example, acidic amino acids elute prior to the void volume of a cation exchange column, since electrostatic repulsion excludes them from most or all of the volume inside the pores of the stationary phase. However, if the mobile phase contains greater than 60% organic solvent, then acidic amino acids are retained almost as well by a cation exchange column as by a neutral column (1). With sufficient organic solvent in the mobile phase, hydrophilic interaction overcomes electrostatic effects and dominates the chromatography.

Another example of electrostatic effects in HILIC is the separation of phosphoproteins. Histone proteins are basic and are well-retained on a cation-exchange column. Any phosphate groups attached to the histone protein repel the column electrostatically, leading to earlier elution of the protein. However, a high level of organic solvent in the mobile phases induces hydrophilic interaction of the phosphate groups with the column. This leads to later elution of phosphorylated histones despite the electrostatic repulsion [32].

Electrostatic effects have important implications for the separation of charged solutes by HILIC. Normally, basic solutes are the best retained in HILIC, followed by phosphorylated ones (1). Therefore, a gradient is necessary to elute samples containing very basic peptides or nucleotides such as ATP. In extreme cases, a gradient is required with both decreasing organic and increasing salt concentrations [8]. The use of gradients is more complicated than isocratic elution and involves additional equipment. If isocratic elution is used, much longer elution profiles may be needed. Furthermore, ineffective separation may result if appropriate gradient elution conditions are not selected. There is a need in the art for methods which permit the isocratic and rapid resolution of certain charged solutes.

### SUMMARY OF THE INVENTION

In one aspect, a method of performing electrostatic repulsion-hydrophilic interaction chromatography (ERLIC) on a protein, peptide, or amino acid comprises providing a column having an anion-exchange material at a pH of less than about 4, and eluting the compound using a mobile phase comprising an amount of organic solvent sufficient to confer hydrophilic interaction that substantially balances the electrostatic repulsion by the stationary phase. The method is effective, for example, for the selective isolation of phosphopeptides, either isocratically or using a salt gradient. In another aspect, a method of performing ERLIC on a nucleic acid or nucleotide comprises providing a column having an cation-exchange material at a pH of less than about 3.4, and eluting the compound using a mobile phase comprising organic solvent sufficient to confer hydrophilic interaction that substantially balances the electrostatic repulsion by the stationary phase.

### BRIEF DESCRIPTION OF THE DRAWINGS

The objects, features, and advantages of the invention will be apparent from the following more detailed description of certain embodiments of the invention and as illustrated in the accompanying drawings in which:

FIG. 1 illustrates a typical separation of peptide standards in the anion-exchange mode. Gly-Tyr is a neutral peptide that elutes in the void volume. The moderately acidic peptides Asp-Val and Ala-Gly-Ser-Glu are retained to some extent by electrostatic attraction. However, in the absence of hydrophilic interaction the basic peptide [Arg⁸]-vasopressin is excluded from the pore volume by electrostatic repulsion and elutes prior to the void volume.

FIG. 2 shows the retention time of several standard peptides as a function of acetonitrile concentration using the HILIC method.

FIG. 3 shows the retention time of several standard peptides versus acetonitrile concentration using the ERLIC method.

FIG. 4 compares the separation of a mixture of peptide standards using the HILIC and ERLIC methods. The top half of the figure shows the elution profile using the HILIC mode, and the bottom half of the figure shows the elution profile using the ERLIC mode.

FIG. 5 demonstrates the effect of the pH of the mobile phase on peptide retention time using ERLIC.

FIG. 6 demonstrates the effect of the salt concentration of the mobile phase on peptide retention time using ERLIC.

FIG. 7 illustrates chromatography of the tryptic digest of beta-casein. Digestion of the protein beta-casein with trypsin yields about 14 fragments. One of these peptides has one phosphate group and another has four. The top chromatogram shows this digest being run under ERLIC conditions on a PolyWAX LP™ column (an anion-exchange material) at pH 2.0. Under these conditions, carboxyl-groups in the peptides have lost their negative charge. The second and third chromatograms show the elution of authentic standards of the phosphopeptides in the digest.

FIG. 8 contrasts the migration of phosphopeptides on an anion-exchange column (as in FIG. 7) in the ERLIC mode with their migration on the same column used in the ordinary anion-exchange (AEX) mode. The conditions were identical except that in the AEX mode both mobile phases contained just 10% acetonitrile, not nearly enough to confer hydrophilic interaction on the chromatography, so the peptide with one phosphate is poorly separated from peptides with no phosphate group.

FIG. 9 illustrates a set of synthetic peptides with the same amino acid sequence. They differ in having 0, 1, 2, 3 or 4 phosphate groups on the serine residues. The insert also shows the separation of positional variants: two peptides with the same number of phosphates (2) but on different serine residues. Again, the peptide with one phosphate is well-retained in the ERLIC mode but not in the AEX mode.

FIG. 10 shows retention as a function of triethylamine methylphosphonate concentration in ERLIC of amino acids.

FIG. 11 shows the effect of triethylamine phosphate concentration of the mobile phase on retention of amino acids in ERLIC.

FIG. 12 illustrates the isocratic elution of acidic amino acids in the same time frame as basic amino acids in the ERLIC mode.

FIG. 13 shows the effect of increasing acetonitrile concentration in the mobile phase from 65 to 70% on retention times of amino acids. With increasing hydrophilic interaction, retention times of basic amino acids increased to the point that electrostatic repulsion no longer sufficed to cause their isocratic elution in the same time frame as the other amino acids.

FIG. 14 shows the effects of salt concentration, acetonitrile concentration, and pH on the retention of amino acids in HILIC. Isocratic conditions that lead to adequate separation of the nonbasic amino acids cause the basic amino acids to elute in a later time frame.

FIG. 15 compares ERLIC of nucleotides on a cation-exchange column, PolySULFOETHYL Aspartamide™, with HILIC of these compounds on a column of a neutral material, PolyHYDROXYETHYL A™. At low concentrations of ACN where hydrophilic interactions are negligible, ADP elutes earlier than AMP from the cation-exchange column due to its greater electrostatic repulsion. At higher levels of ACN, where hydrophilic interactions with the phosphate groups become significant, their elution order is reversed.

FIG. 16 illustrates that at pH 6, where phosphate groups are beginning to acquire their second negative charge, electrostatic repulsion is so great that no nucleotide or oligonucleotide is retained in ERLIC. Retention increases with decreasing pH, particularly below 3.4 where the phosphate groups begin to lose their single negative charge.

FIG. 17 shows the effect of the base on retention in ERLIC, in particular U˜T<A<G<C. At the ACN level used here, phosphorylation promotes retention in every case.

FIG. 18 shows the results obtained with TEA-MePO₄ substituted for TEAP. There is an increase in sensitivity to the number of phosphate groups at the expense of sensitivity to the base involved.

FIG. 19 illustrates isocratic elution of nucleotides in ERLIC with a mobile phase of 80 mM TEAP, pH 3.0, 84% acetonitrile.

FIG. 20 is a schematic illustration of the orientation of amino acids in ERLIC. With phosphate as the counterion, its potential second negative charge provides a means for the attraction of basic amino acids to the surface.

FIG. 21 is a schematic contrasting the orientation of AMP and ATP. The phosphate group of AMP, being quite hydrophilic, is oriented toward the stationary phase despite being repelled by it. In the case of ATP, the electrostatic repulsion between the three phosphate groups and the surface is sufficient to orient the phosphate groups away from the surface.

### DETAILED DESCRIPTION OF THE INVENTION

The present invention introduces a new strategy of performing chromatography to separate mixtures with highly charged solutes which are either too poorly or too avidly retained during HILIC. The strategy involves combining the principles of ion exchange and hydrophilic interaction. Using the methods of the present invention, amino acids, peptides, nucleic acids or nucleotides that would not be retained or would be retained too well by the stationary phase in either HILIC or ion-exchange chromatography can be effectively separated in a reasonable time frame. The methods superimpose the separation power of both hydrophilic interaction and electrostatic interaction, thereby antagonizing the extremes of retention found with either method alone. By eliminating both long and short retention times for certain solutes, the methods permit isocratic resolution of heterogeneous mixtures which otherwise would require complex gradient elution. This new combination of chromatography principles is termed electrostatic repulsion-hydrophilic interaction chromatography (ERLIC).

The ERLIC method involves matching the appropriate mobile phase to the stationary phase and the sample being separated or analyzed in order to resolve the sample isocratically and rapidly. ERLIC applies the following two principles to achieve this result: (1) The stationary phase should possess the same polarity of charge as the majority of the compounds in the sample. This provides electrostatic repulsion in order to prevent undue retention of highly charged solutes that are strongly retained by the stationary phase through hydrophilic interaction. (2) If the electrostatic repulsion for one or more solutes is too great, causing them to elute too soon (e.g., before the void volume), then the organic solvent content of the mobile phase should be increased. This will increase the retention time of rapidly eluting solutes, e.g., those with multiple charges of the same polarity as the stationary phase, by increasing the strength of hydrophilic interactions. This can be accomplished, for example, by raising the concentration of acetonitrile or propanol in the mobile phase. The retention time of such solutes can be increased over a wide range. Preferably, the retention time is reduced so as to shorten the overall elution time for a sample or lengthened to improve the ability of the method to isocratically resolve the components in a sample. Most preferably, the retention time of multiply charged solutes is decreased to cause their elution within the same time frame as the other compounds in the sample.

### General Applications for ERLIC

Using general isocratic conditions, ERLIC is capable of providing separation or analysis of a variety of small molecules or macromolecules which normally would require gradients. ERLIC can generally be applied to any compound with sufficient polarity to be retained by the stationary phase under the conditions of HILIC (e.g., using a mobile phase comprising 10%-40% water by volume and a stationary phase material more polar than the mobile phase such that the compound elutes at a volume greater than the void volume). ERLIC is equally well suited to the analysis of individual compounds and to the separation of compounds from a mixture. In the analytical mode, a compound can be identified based on its elution position compared to a standard, or the purity and composition of a mixture of compounds can be assessed by the overall elution profile of the mixture. In the preparative mode, ERLIC can be applied to the isolation or purification of a particular compound from a mixture by physically separating individual compounds, which correspond to distinct peaks in the elution profile, as they elute from the column. ERLIC can be applied to the separation or analysis of mixtures of amino acids, peptides, polypeptides, proteins, nucleotides, oligonucleotides, or polynucleotides.

The ERLIC method can simplify the development of chromatographic separations considerably by allowing many HILIC separations to be performed isocratically. Not all mixtures will lend themselves to such treatment, however. Complex mixtures containing large numbers of individual molecules, e.g., a protein digest containing over 50 peptides, probably cannot be entirely separated using a single isocratic procedure. However, complete separation is not always necessary, particularly in the analytical mode. For example, if a mass spectrometer is used as the detector, it is only necessary to reduce the number of peptides coeluting as a single peak or unresolved group of peaks to an extent that the ionization of each peptide is not interfered with by the presence of other peptides. An automatic sample injector can be used to analyze a large number of samples rapidly with each sample being injected after the ERLIC elution window of the preceding sample. The use of isocratic elution also simplifies equipment needs, since gradient elution usually requires an additional pump and a gradient-forming device. Moreover, ERLIC can be useful for separations performed on a silicon wafer or chip, in which many samples might be analyzed simultaneously on a minute scale in separate channels. Flow rates for such applications can be on the order of nanoliters per minute. The equipment needed for such separations would be greatly simplified if they could be performed isocratically.

### Selection of the Stationary Phase

Just as for HILIC, in ERLIC the stationary phase is composed of a hydrophilic material. In addition, in ERLIC the stationary phase material must also be either positively or negatively charged at the pH of the mobile phase. Specific materials are provided in the Examples.

### Selection of the Mobile Phase

As with HILIC, the mobile phase in ERLIC is less polar (more hydrophobic) than the stationary phase. The mobile phase should contain at least 2% water by volume so that the stationary phase material can form a stagnant layer of bound water, which is instrumental in retaining more polar solutes longer than less polar ones. Generally, the mobile phase will contain about 40% to 90% by volume of an organic solvent such as acetonitrile, methanol, propanol, or another solvent with similar polarity which is miscible with water. The concentration of the organic solvent can be adjusted as desired to alter the retention of a compound of interest. The pH of the mobile phase is an important factor in setting the net charge of the stationary phase and the solutes, which also affects the retention of a compound of interest.

### Strategies for Adjusting the Elution of a Compound in ERLIC

The ERLIC method is particularly well suited to address several extremes of retention encountered in HILIC. As described generally above, the HILIC component of ERLIC can shift early eluting molecular species to a later elution time, resulting in better resolution. With appropriate adjustments to the mobile phase, the electrostatic repulsion component of ERLIC can also cause late eluting molecular species to elute earlier, which shortens run time with little or no negative impact on resolution. The following cases are illustrative.

### Late Elution of Very Acidic Peptides Through Electrostatic Attraction

Highly acidic peptides may be retained excessively during anion exchange chromatography using HILIC due to electrostatic attraction to the stationary phase. One solution to this problem is the use of a mobile phase with sufficiently low pH to uncharge aspartate and glutamate residues, leaving most peptides neutral or basic. Using the isocratic methods of the present invention, the organic solvent content of the mobile phase can be increased to such a level that hydrophilic interaction dominates the chromatography and insures retention of the acidic peptides despite the lack of electrostatic attraction.

### Late Elution of Very Basic Peptides Through Hydrophilic Interaction

Highly basic peptides elute late from polar columns in the HILIC mode. The use of electrostatic repulsion by the stationary phase, i.e., the ERLIC method, would allow such peptides to elute within the same time frame as neutral or moderately acidic peptides. This effect would be analogous to having an immobilized salt gradient.

### Elution of Basic Peptides Prior to the Void Volume Through Electrostatic Repulsion

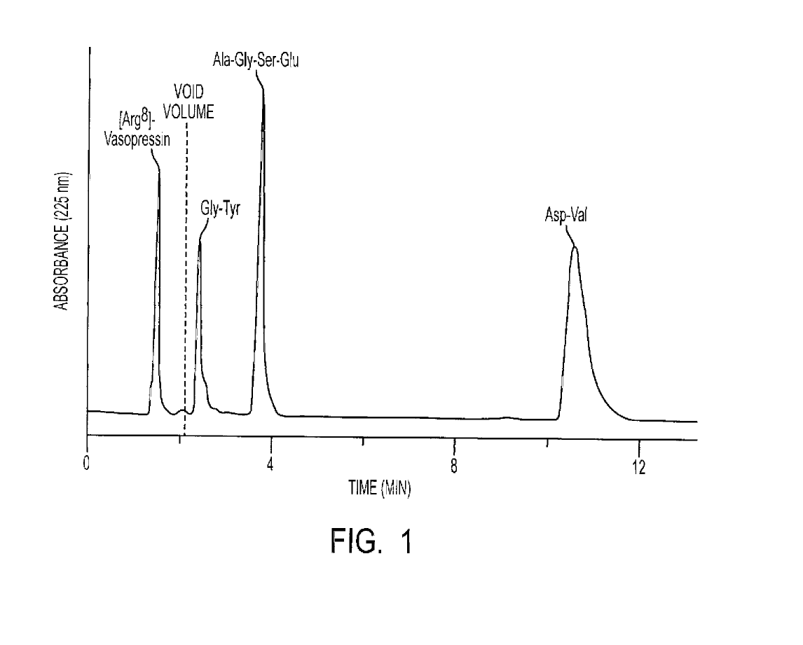

If peptides are separated in the AEX mode using a positively charged stationary phase, basic peptides will elute in or before the void volume due to electrostatic repulsion effects (see FIG. 1). This results in a narrow fractionation range which is of limited utility. However, if sufficient organic solvent is included in the mobile phase, hydrophilic interaction will be strong enough to overcome the electrostatic repulsion effect, resulting in longer, more reasonable retention times and improved resolution.

Acidic amino acids elute prior to the void volume of a cation-exchange column, since electrostatic repulsion denies them access to the full pore volume of the stationary phase. However, if the mobile phase contains >60% organic solvent, then acidic amino acids are retained almost as well by a polar cation-exchange column as by a polar neutral column [1]. This seeming anomaly reflects the fact that hydrophilic interaction is independent of electrostatic effects. With sufficient organic solvent in the mobile phase, hydrophilic interaction dominates the chromatography. Thus, phosphate groups decrease the retention of basic histone proteins on a cation-exchange column in the absence of organic solvent but lead to a net increase in retention if the mobile phase contains 70% ACN [32]. Under these conditions, the hydrophilic interaction conferred by the phosphate groups is stronger than their electrostatic repulsion by the stationary phase.

This phenomenon has important implications. Basic solutes are normally the best-retained in HILIC, followed by phosphorylated ones [1]. A gradient is necessary if samples contain very basic peptides or nucleotides such as ATP. In extreme cases, a gradient is required with both decreasing organic and increasing salt concentrations [8]. However, in the case of peptides, this could conceivably be unnecessary if an anion-exchange column were used for HILIC. This combination would address the following three extremes of retention (1) late elution of very acidic peptides through electrostatic attraction: this could be moderated by use of mobile phases with pH low enough to uncharge aspartate and glutamate residues, leaving most peptides neutral or basic. In fact, a decreasing pH gradient has been used with anion-exchange cartridges to release trapped acidic peptides [41] and for desalting proteins [42]; (2) Late elution of very basic peptides through hydrophilic interaction: electrostatic repulsion by the stationary phase would throw such peptides back into the elution time frame of neutral or moderately acidic peptides. The effect would be analogous to having an immobilized salt gradient; (3) Elution of basic peptides prior to the void volume through electrostatic repulsion: as with acidic amino acids, in the absence of a high level of organic solvent, basic peptides are excluded from the pore volume of a column of the same charge (FIG. 1). This is a version of ion-exclusion chromatography [43-45], a technique of limited utility because of its narrow fractionation range. However, one could include enough organic solvent in the mobile phase to generate hydrophilic interaction sufficient for reasonable retention of such peptides.

Under these conditions, all peptides in a mixture would be retained through hydrophilic interaction despite being repelled by the stationary phase to some extent (except for neutral peptides). The acronym ERLIC is proposed for this combination, standing for Electrostatic Repulsion-Hydrophilic Interaction Chromatography. Since the two superimposed modes antagonize each other's extremes of retention, isocratic resolution of heterogeneous peptide mixtures may be practical.

Peptides that contained phosphate or sulfate groups would retain some negative charge even at a pH low enough to uncharge Asp- and Glu-residues. Such peptides would display some electrostatic attraction to the stationary phase used for ERLIC. This would be an asset rather than a liability; numerous applications in biochemistry would benefit from a method permitting the selective isolation of phosphopeptides from a digest. In this ERLIC represents an alternative to Immobilized Metal Affinity Chromatography (IMAC) and Lewis acids such as titania, zirconia or alumina. In cases where a peptide contained more than one phosphate or sulfate group, elution with a salt gradient may still be necessary.

In addition to peptides, ERLIC could in principle be applied to other solutes with sufficient charge, either positive or negative. This study explores the characteristics and utility of ERLIC as applied to peptides, amino acids, nucleotides and oligonucleotides.

### Materials and Methods

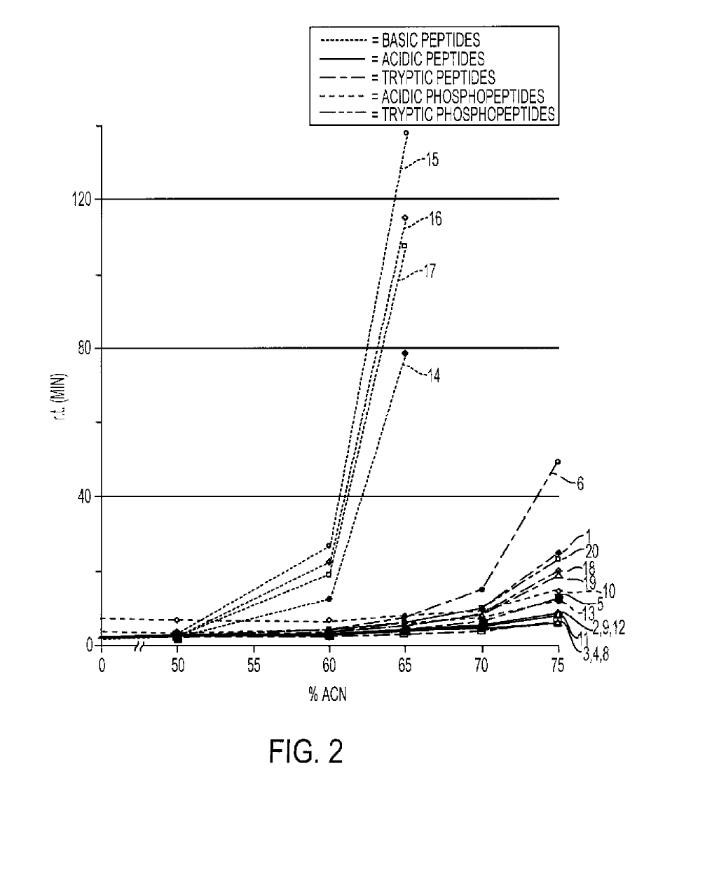

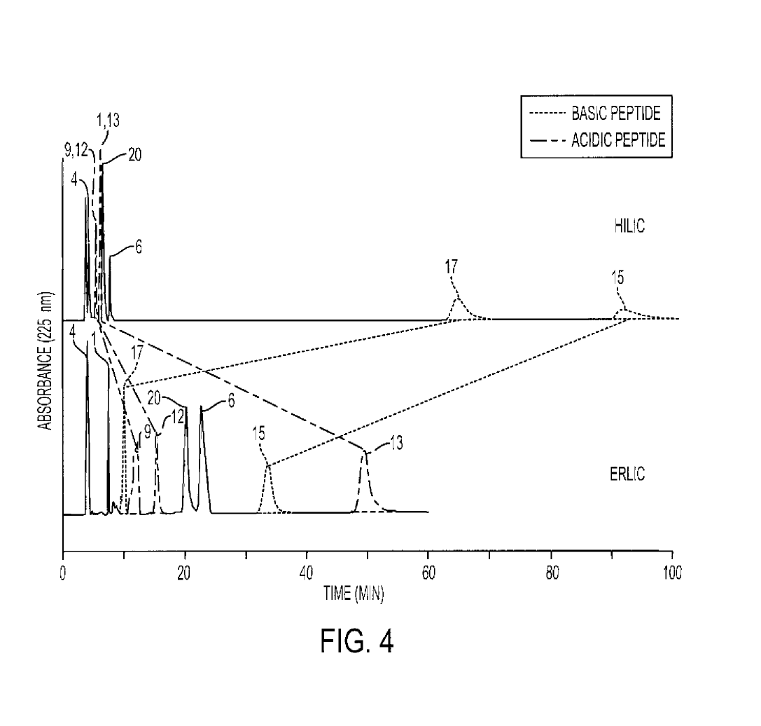

All columns were products of PolyLC Inc. (Columbia, Md.) except as noted below. PolyWAX LP™, a weak anion-exchange material, was used with peptides and amino acids. For peptides, the columns were either: 1) 100×4.6-mm, 5-μm particle diameter, 300-Å pore diameter (item#104WX0503), or 2) 200×4.6-mm, 5-μm, 300-Å (item#204WX0503). For amino acids, the column was 200×4.6-mm, 5-μm, 100-Å (item#204WX0501). For ERLIC of nucleotides, a 200×4.6-mm column of the strong cation-exchange material PolySULFOETHYL Aspartamide™ (PolySULFOETHYL A™) [46] was used; 5-μm, 300-Å (item#204SE0503). HILIC data for peptides (FIGS. 2 and 4), nucleotides and nucleic acids was obtained with a 200×4.6-mm column of PolyHYDROXYETHYL Aspartamide™ (PolyHYDROXYETHYL A™) [1]; 5-μm, 300-Å (item#204HY0503). HILIC data for amino acids was obtained with a 200×4.6-mm column of 5-μm, 100-Å PolyHYDROXYETHYL A™ (item#204HY0501).

Equipment: A Scientific Systems Inc./Lab Alliance (State College, Pa.) Essence HPLC system was used.

Reagents: peptide standards 1-20 were purchased from Bachem (Torrance, Calif.), with the following exceptions: 9 (Sigma Chemical Co., St. Louis, Mo.); 15, 16 (Peninsula Laboratories, Belmont, Calif.); and 13, 18-20 (California Peptide Research, Napa, Calif.). Amino acid, nucleotide and nucleic acid standards were from Sigma. Phosphoric acid and acetonitrile (ACN) [both HPLC-grade] were from Fisher Scientific (Pittsburgh, Pa.). Triethylamine (99.5%) was from Aldrich Chemical Co. (Milwaukee, Wis.). Methylphosphonic acid was from Alfa Aesar/Lancaster Synthesis (Ward Hill, Mass.). HPLC-grade water was used.

0.5 M stock solutions of triethylamine phosphate (TEAP) buffers were prepared as follows: 14.4 g. of 85% phosphoric acid was weighed into a beaker and 150 ml of water added slowly, with stirring. Triethylamine was added (in a hood) until the desired pH was attained. The solution was diluted to 250 ml and filtered (0.45-μm filter). Methylphosphonate stock solutions were prepared using the same procedure, with addition of either triethylamine or NaOH solution. Mobile phases were prepared from water, ACN, and aliquots of the stock solutions. The pH of the mobile phases was neither measured nor adjusted, since dissociation constants shift in predominantly organic solution [47], but was merely designated with the pH of the stock solution used to prepare them.

### Peptide Standards:

| MODEL TRYPTIC PEPTIDES | |
|---|---|
| 1) | Thr-Tyr-Ser-Lys |
| 2) | Asp-Leu-Trp-Gln-Lys (Uremic pentapeptide) |
| 3) | Tyr-Gly-Gly-Phe-Leu-Arg (Dynorphin A (1–6), porcine) |
| 4) | Leu-Val-Val-Tyr-Pro-Trp-Thr-Gln-Arg (Leu-Valorphin-Arg) |
| 5) | Phe-Ser-Trp-Gly-Ala-Glu-Gly-Gln-Arg (Experimental Allergic Encephalitogenic |
| | Peptide) |
| 6) | Val-Gln-Gly-Glu-Glu-Ser-Asn-Asp-Lys (β-interleukin (163–171), human) |
| ACIDIC PEPTIDES | |
| 7) | Asp-Val |
| 8) | Val-Asp |
| 9) | Glu-Ala-Glu |
| 10) | Asp-Ala-Asp-Glu-(pTyr)-Leu (EGF receptor (988–993), human (phosphorylated) |
| 11) | Trp-Ala-Gly-Gly-Asp-Ala-Ser-Gly-Glu (DSIP; Delta Sleep-Inducing Peptide) |
| 12) | “ “isoAsp” “([isoAsp⁵]-DSIP) |
| 13) | “ “ pSer ” “([phosphoSer⁵]-DSIP) |
| BASIC PEPTIDES | |
| 14) | ACTH (1–39; human) [6 acidic and 7 basic residues] |
| 15) | Arg-Lys-Arg-Ser-Arg-Lys-Glu |
| 16) | Lys-Arg-Gln-His-Pro-Gly-Lys-Arg (TRH Precursor Peptide) |
| 17) | Lys-Pro-Val-Gly-Lys-Lys-Arg-Arg-Pro-Val-Lys-Val-Tyr-Pro |
| MODEL DSIP-LIKE TRYPTIC PEPTIDES | |
| 18) | Trp-Ala-Gly-Gly-Asp-Ala-Ser-Gly-Lys |
| 19) | “ “isoAsp” “ |
| 20) | “ “ pSer ” “ |

### ERLIC of Peptides

A. Selection of Standards

1) Tryptic peptides. With the exception of those containing His- residues or missed cleavages, tryptic peptides contain only two basic groups, the N-terminus and the C-terminal Arg- or Lys- residue. This would simplify the analysis since no unduly basic peptides would be present. Therefore, a number of tryptic peptides were included in the standards to get some idea of the range of elution times that could be expected. Standards 1-5 are ordinary sequences with 0 or 1 acidic residues. Standard 6 is an unusually acidic tryptic sequence. Standards 18-20 are tryptic sequences substituted with an Asp-, isoAsp- or phosphoSer-residue at one position; this sample set permits an assessment of the effects of these residues on retention.

2) Acidic peptides. Standards 7-9 are acidic peptides with no basic groups except for the N-terminus. Standard 10 is an unusually acidic phosphopeptide, as is frequently the case with sequences surrounding phosphoTyr-residues. Standards 11-13, the DSIP peptides, have the same sequences as standards 18-20 except for substitution of a Glu- residue for the C-terminal Lys-. This permits the assessment of the effects of replacing an Asp- with an isoAsp- or phosphoSer- residue although in a less controlled fashion than with standards 18-20, since the residue being substituted is not the only acidic residue in the peptide.

3) Basic peptides. Standards 15-17 are unusually basic peptides. Standard 14, ACTH, has almost as many acidic as basic residues. This could help in assessing the relative importance of such residues in the overall retention of a peptide.

TABLE 1

| | RETENTION TIME (MIN.) | | | |
|---|---|---|---|---|
| | IN MOBILE PHASE INDICATED | | | |
| | 20 mM | 50 mM | 20 mM | 50 mM |
| STANDARD | TEAP | TEAP | Na—MePO₄ | Na—MePO₄ |
| 1 | 3.5 | 4.6 | 2.3 | 2.8 |
| 2 | 2.7 | 3.2 | 2.1 | 2.4 |
| 3 | 2.4 | 2.7 | 1.8 | 2.0 |
| 4 | 2.3 | 2.6 | 1.8 | 1.9 |
| 5 | 2.6 | 3.1 | 2.0 | 2.1 |
| 6 | 4.3 | 5.4 | 3.4 | 3.8 |
| 7 | 3.2 | 3.2 | 3.6 | 3.4 |
| 8 | 3.2 | 3.4 | 3.4 | 3.2 |
| 9 | 3.5 | 3.7 | 3.9 | 3.9 |
| 10 | 10.5 | 6.0 | 64.0 | 24.5 |
| 11 | 3.1 | 3.2 | 3.4 | 3.1 |
| 12 | 3.5 | 3.4 | 5.1 | 4.0 |
| 13 | 6.5 | 4.7 | 19.0 | 11.2 |
| 14 | (long) | (long) | 1.7 | 1.9 |
| 15 | (long) | (long) | 2.3 | 3.4 |
| 16 | 16.5 | 17.4 | 2.1 | 3.1 |
| 17 | 6.9 | 22.6 | 1.8 | 2.3 |

B. Selection of Mobile Phases

Table 1 shows a preliminary comparison of retention times with TEAP vs. sodium methylphosphonate (Na-MePO₄) buffers. With TEAP buffers, retention of acidic phosphopeptides (standards 10 and 13) was not markedly greater than that of other peptides, while retention of basic standards 13-17 was greater than most of the others. This selectivity, characteristic of ordinary HILIC, was the opposite of that desired. By contrast, the selectivity of Na-MePO₄ buffers exhibited the characteristics desired of ERLIC, with rapid elution of basic peptides and delayed elution of phosphopeptides. Accordingly, Na-MePO₄ buffers were selected for detailed examination of ERLIC of peptides.

C. Effect of % Organic Solvent; HILIC vs. ERLIC

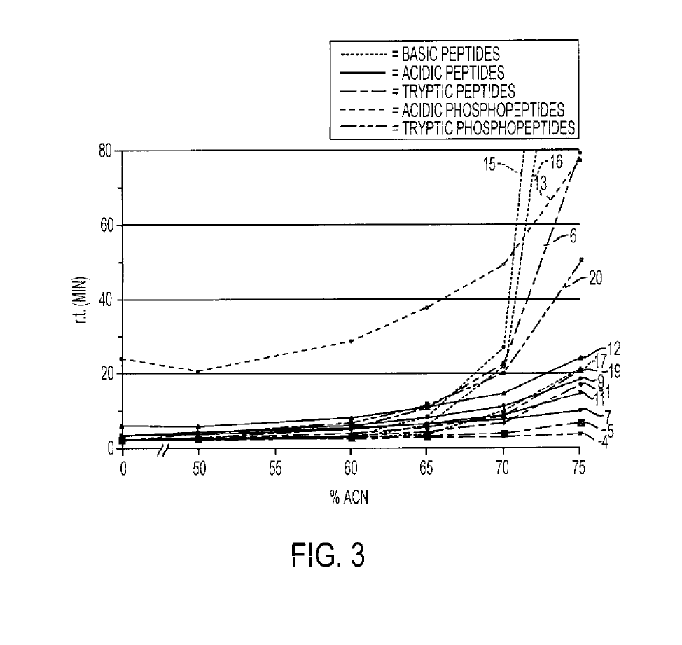

FIG. 2 shows the effect of % ACN on the isocratic retention of peptide standards 1-20 in HILIC. Basic peptides (standards 14-17) are by far the best-retained. All the others, including phosphopeptides, elute roughly in the same time frame. FIG. 3 shows the same standards under ERLIC conditions. Owing to the electrostatic repulsion, basic peptides now elute in the same time frame as the other peptides at ACN levels of 70% or below. This contrast between HILIC and ERLIC is manifest in FIG. 4, which compares chromatograms of a peptide standard set run in both modes. In the HILIC mode, a level of ACN that leads to isocratic elution of basic standards 15 and 17 in less than 100′ affords inadequate retention of the acidic and neutral standards. In the ERLIC mode, the electrostatic repulsion of 15 and 17 now permits the concentration of ACN to be increased to a level that affords adequate retention and isocratic elution of all standards in this example within 50′.

Evidently the concentration and pH of the mobile phase in FIG. 3 suffices to suppress the ionization of carboxyl- groups, since acidic standards 7-9 and 11 elute in the same time frame as the neutral tryptic peptides. The acidic tryptic peptide 6 is retained significantly longer than other tryptic peptides. Judging from FIG. 1, this reflects its hydrophilicity as well as its acidity. IsoAsp-containing peptides elute somewhat later than their Asp- containing analogs (e.g., 12 vs. 11). Between 65-70% ACN, the phosphorylated standard 20 is the last or nearly the last tryptic peptide to elute. Acidic phosphopeptide 13 elutes much later than the other standards under these conditions, while the even more acidic phosphopeptide standard 10 does not elute at all in a reasonable time frame. This emphasizes the importance of the role played by the second basic residue in a tryptic fragment, and the attendant electrostatic repulsion, in assuring elution in a reasonable time frame in ERLIC without the use of high levels of salt. The significant increase in retention of phosphopeptides at high levels of ACN reflects the great hydrophilicity of phosphate groups which is superimposed on their electrostatic attraction. However, at ACN levels above 70%, hydrophilic interactions become so strong that some basic standards (15 and 16) once again become the best-retained peptides despite the electrostatic repulsion. This seems to define the window of ACN concentration for selective isolation of phosphopeptides from digests. Of course, in tryptic digests with no missed cleavages, no peptides will have large numbers of basic residues unless they are crosslinked or contain His-.

FIG. 3 suggests that it is possible to set up a well-defined window of isocratic elution for all peptides in a mixture. The width of the window can be adjusted to some extent by varying the % organic solvent. The composition of the peptides is unimportant as long as none are particularly basic or contain phosphate groups.

D. Effect of pH in ERLIC

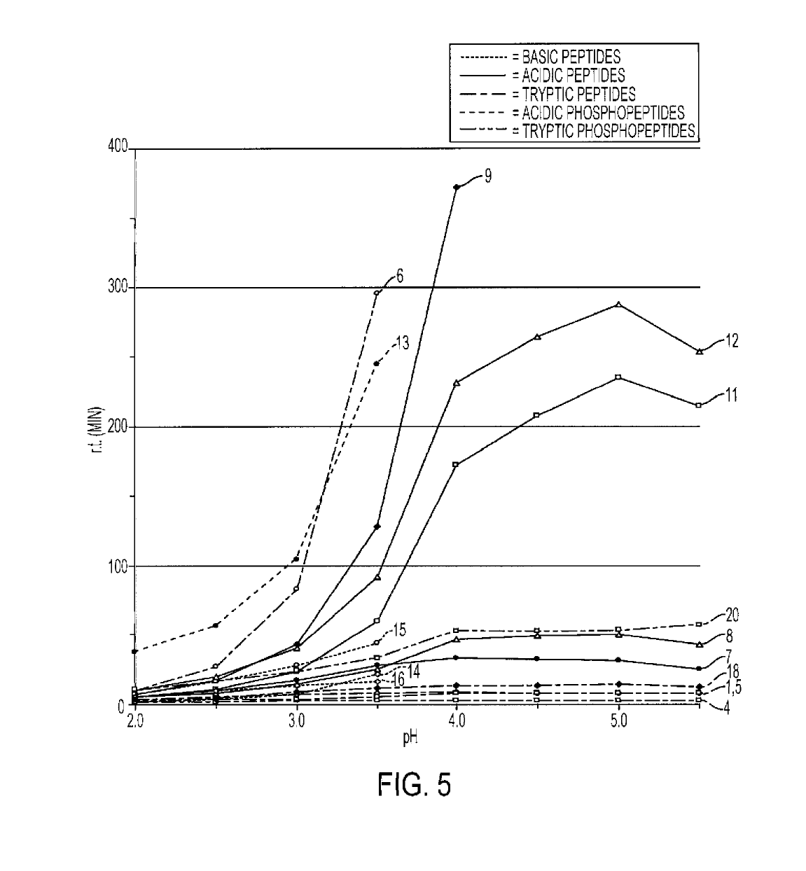

FIG. 5 shows the effect of pH on retention in ERLIC. Since carboxyl- groups are substantially unionized at pH 2.0, the best-retained peptides are phosphopeptide 13 and, to a modest extent, tryptic phosphopeptide 20. As carboxyl- groups ionize at higher pH values, though, retention comes to reflect the total number of acidic groups of all sorts, and the selectivity for phosphopeptides is lost. These conditions converge upon those of ordinary anion-exchange chromatography. Neutral tryptic peptides are retained almost entirely through hydrophilic interactions. Their retention is little affected by pH as long as the mobile phase contains a reasonable concentration of salt. Retention of acidic peptides reaches a maximum at pH 5.0 and then falls off at higher pH values. This reflects a decrease in the charge density of the weak anion-exchange (WAX) material. Titration curves of suspensions of such materials reveal a continuous increase in charge density from pH 9.5 to pH 5.0 [48].

E. Effect of Salt Concentration in ERLIC

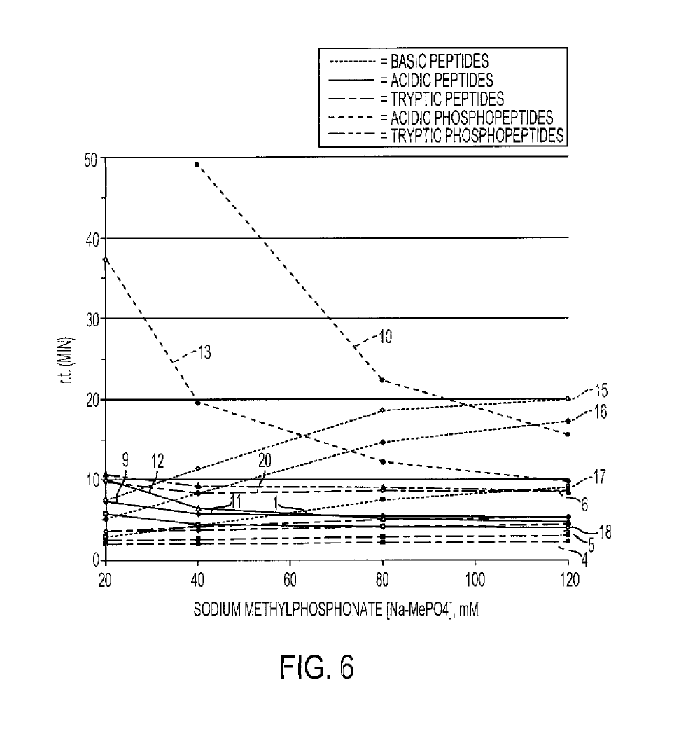

FIG. 6 demonstrates the importance of this variable in determining selectivity. Increasing levels of salt shield solutes from all electrostatic effects, both attractive and repulsive, and the selectivity converges on that of HILIC. Thus, retention decreases for acidic peptides and increases for basic ones, to the point that basic peptides once again become the best-retained at high salt levels. There is a modest increase in retention of neutral tryptic peptides with increasing salt. Presumably this reflects the decreasing repulsion of their N-termini and the basic residues at their C-termini.

This data supplements that in FIG. 3 in setting up conditions for a well-defined window of elution of all peptides in a mixture. With enough salt in the mobile phase, even peptides with numerous basic or phosphate groups will elute in a well-defined time frame.

F. Selective Isolation of Phosphopeptides

The preceding data suggested that peptides with a single phosphate group were likely to be the last or among the last peptides to elute when a tryptic digest was eluted with 20 mM Na-MePO4, pH 2.0, containing 70% ACN. Tryptic peptides with more than one phosphate group proved to require gradient elution. A gradient was selected involving increasing salt and modestly decreasing ACN concentration. The salt chosen for the gradient was TEAP, which is more effective than Na-MePO₄ at eluting phosphopeptides (Table 1).

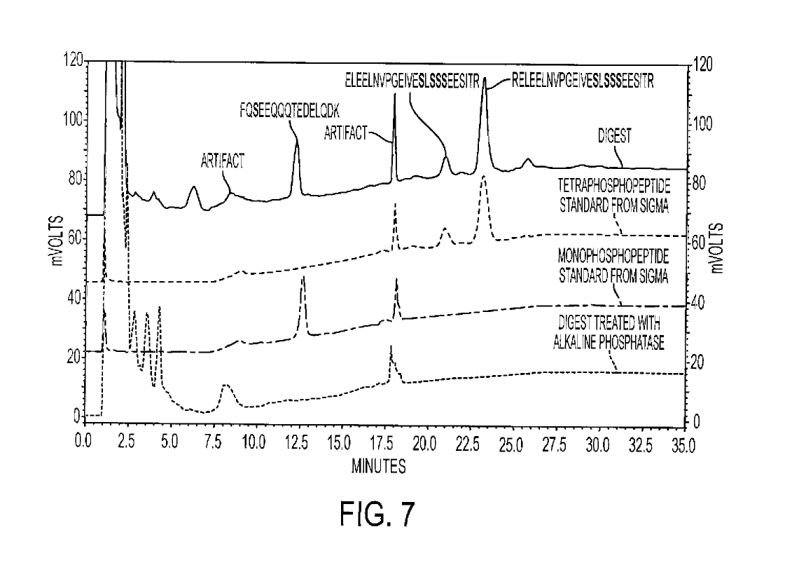

FIG. 7 shows the tryptic digest of the protein beta-casein which contains about 14 fragments. One of these peptides has one phosphate group and another has four. The top chromatogram shows this digest being run on a PolyWAX LP™ column (an anion-exchange material) at pH 2.0. Under these conditions, carboxyl- groups in the peptides have lost their negative charge. There is electrostatic attraction between the positively-charged column material and the negatively-charged phosphate groups in the two phosphopeptides. There is also electrostatic repulsion between the column material and the positively-charged amino-terminus and the lysine or arginine residue at the C-terminus of all tryptic peptides (trypsin cleaves on the C-terminal side of arginine or lysine residues).

Normally the electrostatic repulsion would outweigh the electrostatic attraction of peptides with just one phosphate group. That has been compensated here by including just enough organic solvent in the mobile phase so that the hydrophilic interaction of the basic groups with the stationary phase pretty well balances the electrostatic repulsion. As a result, tryptic peptides lacking phosphate groups elute in or just after the void volume, while tryptic peptides with phosphate groups are much better-retained, since they have the additional electrostatic attraction to the stationary phase. While peptides with a single phosphate group can be eluted isocratically within a reasonable time, that is not true of peptides with multiple phosphate groups. Accordingly, standardized conditions were used involving a gradient of increasing salt (20-200 mM) and slightly decreasing organic solvent concentration (70-60%). There is also a changeover of salt during the gradient from sodium methylphosphonate, a salt that promotes retention of phosphopeptides, to TEAP, a salt that promotes their elution. Phosphopeptides peaks were collected upon elution and the phosphopeptides identified via mass spectroscopy.

The chromatogram on top shows the two expected phosphopeptides in this digest. The peptide sequences are denoted with the single-letter codes for the amino acids. A highlighted “S” denotes a serine residue with a phosphate group. The tetraphosphopeptide normally listed in this digest is a missed-cleavage sequence; the N-terminal amino acid of beta-casein is arginine, and trypsin generally doesn't cleave basic residues in that position. However, in FIG. 7 the correctly-cleaved tetraphosphopeptide can been seen as well, in a 1:6 ratio with the missed-cleavage fragment. Both tetraphosphopeptides are also evident in the commercial standard (chromatogram #2). The commercial standard of the monophosphopeptide (chromatogram #3) coeluted with the corresponding peak in the whole digest (top).

The chromatogram at the bottom of FIG. 7 shows the digest after treatment with alkaline phosphatase, which hydrolyzes phosphate groups off of proteins and peptides. All three peaks identified here as phosphopeptides disappeared from the region of retained solutes while several new peaks appeared in the nonphosphopeptide region at the beginning. This confirms their identity pre-phosphatase treatment as phosphopeptides.

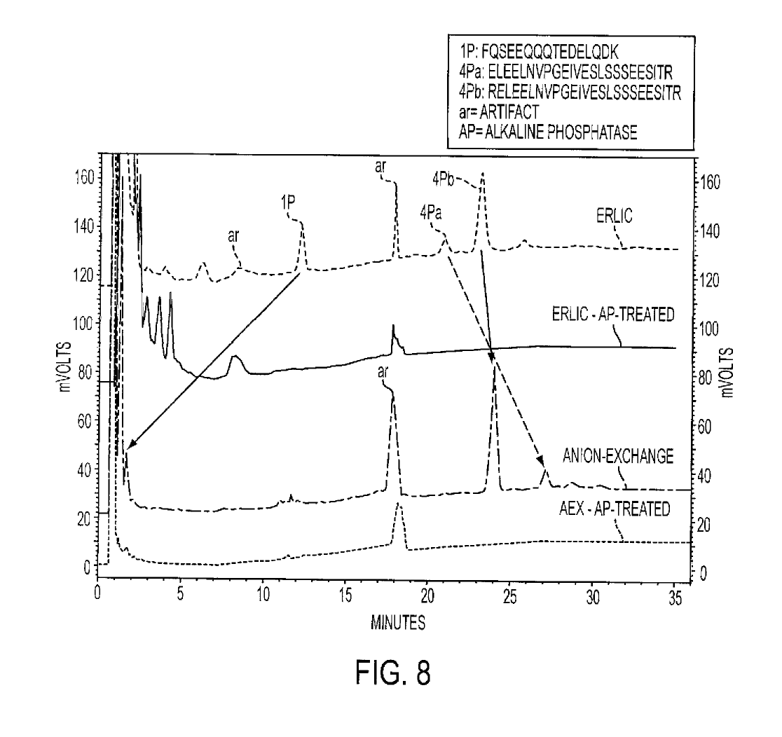

FIG. 8 again shows the tryptic digest of beta-casein. This contrasts the migration of phosphopeptides on an anion-exchange column (same as in FIG. 7) in the ERLIC mode with their migration on the same column used in the ordinary anion-exchange (AEX) mode. The conditions were identical except that both AEX mobile phases contained just 10% acetonitrile, not nearly enough to confer hydrophilic interaction on the chromatography. While the tetraphosphopeptides are still well-retained, the monophosphopeptide now elutes much earlier, among the nonphosphopeptides. Clearly, in the AEX mode the electrostatic repulsion of the two basic groups in the peptide is stronger than the attraction of the single phosphate group. Again, identification of phosphopeptide peaks is confirmed by treating the digest with alkaline phosphatase and running it in both the ERLIC and AEX modes. This data is a clear example that AEX isn't suitable for isolation of singly phosphorylated peptides from tryptic digests, while ERLIC is.

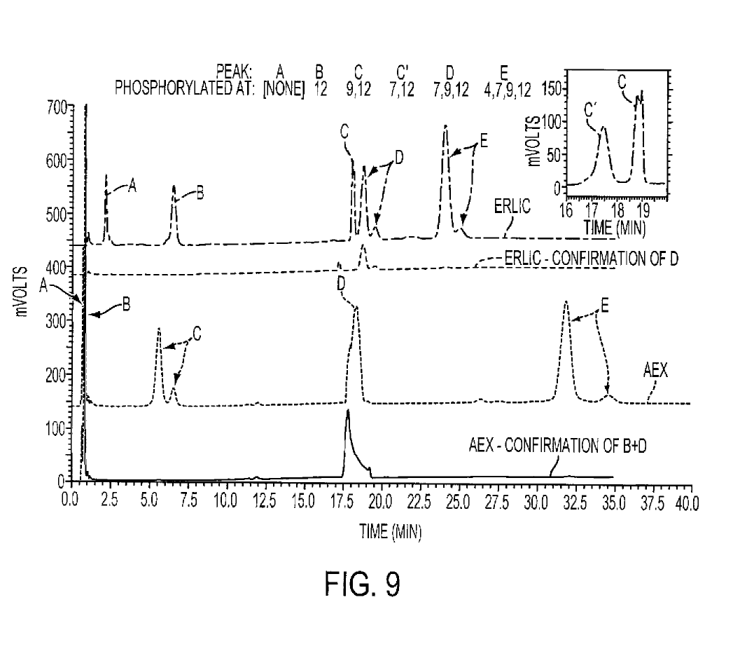

FIG. 9 illustrates synthetic phosphopeptides: This is a set of synthetic peptides with the same amino acid sequence. They differ in having 0, 1, 2, 3 or 4 phosphate groups on the serine residues. The insert also shows the separation of positional variants: two peptides with the same number of phosphates (2) but on different serine residues. Elution conditions were the same as in slides 1 and 2. All peptides were retained and resolved in the ERLIC mode, the phosphopeptides being much better retained than the nonphosphopeptide. In the anion-exchange (AEX) mode, again the peptides with 0 or 1 phosphate elute in or near the void volume, poorly separated.

The doublets observed for some of the peaks probably reflect the separation of cis-trans conformational isomers around the single proline residue. Interconversion between such conformers can be slow relative to the timescale of chromatography and is reported from time to time in the literature. The chromatogram at bottom, with just the B and D standards, shows a peak for D that closely resembles that reported in the literature for a reducing sugar or oligosaccharide [11]. In that case, the two peaks correspond to the alpha- and beta- anomers of the sugars, with a continuum between them corresponding to the molecules interconverting between the two forms during the migration through the HPLC column.

This data demonstrate the utility of ERLIC for the separation of nonphosphopeptides from phosphopeptides, even those with just one phosphate group. By contrast, while anion-exchange (AEX) can be used to isolate peptides with more than one phosphate group, it is not generally useful for peptides with only one phosphate. Such peptides account for the vast majority of peptides in tryptic digests. Thus, ERLIC is promising for research in proteomics, where there is ongoing interest in isolation and identification of phosphopeptides.

### II. ERLIC of Amino Acids

A. Effect of Salt Identity and Concentration on Selectivity

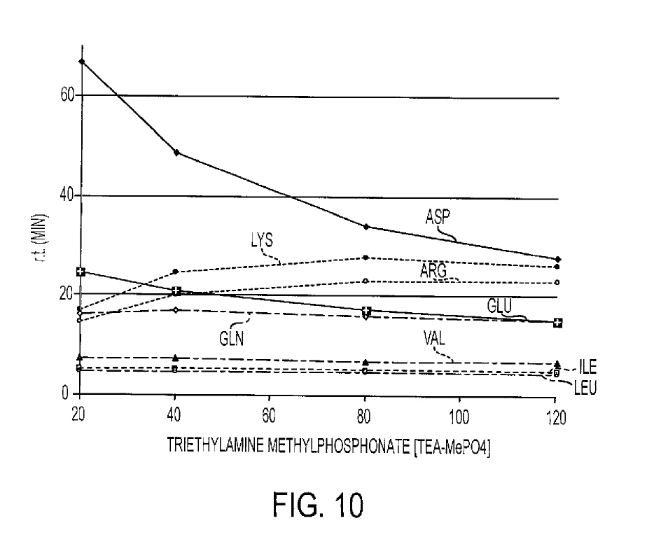

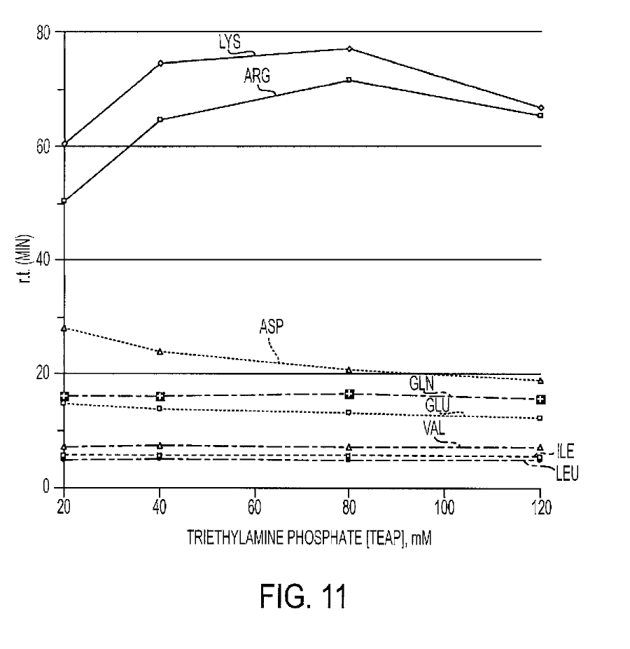

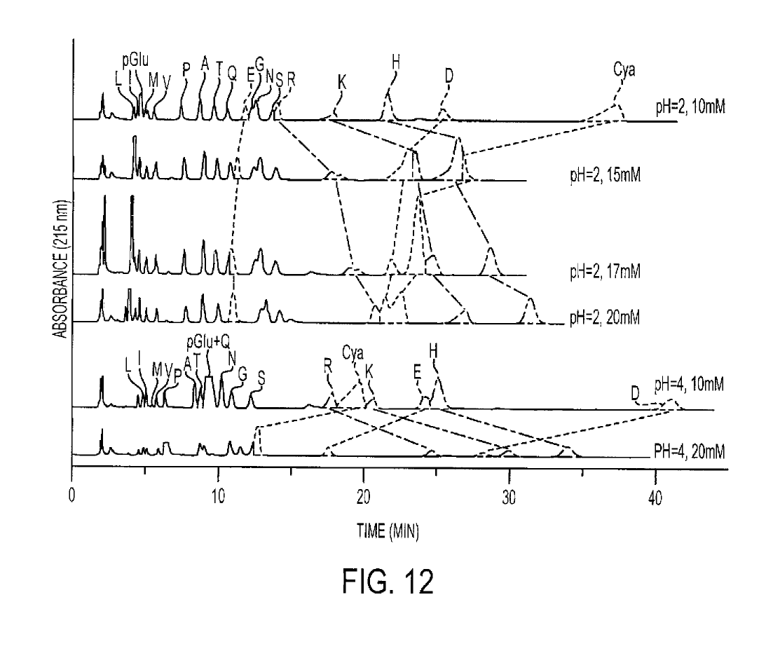

The results in FIG. 10 compare well with those for peptides in FIG. 6; with TEA-MePO4, as with Na-MePO4, there is marked retention of acidic amino acids and comparably early elution of basic ones. Increasing salt concentration suppresses both electrostatic repulsion and attraction, leading to earlier elution of acidic amino acids and later elution of basic ones. There is a slight decrease in retention of neutral amino acids with increasing salt. This reflects the fact that both ERLIC and HILIC are variants of normal-phase chromatography; increasing the polarity of the mobile phase promotes elution. Also, unlike neutral tryptic peptides, neutral amino acids have no marked electrostatic repulsion that would be shielded by the higher salt levels. The results with TEAP (FIG. 11) compare well with those for peptides in Table 1; great retention of basic amino acids and comparably weak retention of acidic ones. However, salt concentrations below 20 mM are apparently too low to maintain a counterion layer, or electrical double layer, that effectively screens the underlying stationary phase. The consequence is that solutes are exposed to more of the positive charge of the stationary phase, so basic amino acids are electrostatically repelled more and elute earlier while acidic ones are attracted and elute later. This permits the isocratic elution of both acidic and basic amino acids in the same time frame (FIG. 12). The retention times of basic and acidic amino acids are extremely sensitive to the electrolyte concentration within the range of 10-20 mM; higher salt levels shield basic amino acids from electrostatic repulsion and cause them to elute later, while the shielding decreases electrostatic attraction of acidic amino acids and causes them to elute earlier. Retention times of neutral amino acids are little affected in this range. Under these conditions, Phe-, Trp- and Tyr- were incompletely resolved from Leu-, Ile- and Val-, and so were omitted from the mixture. It should be noted that Gln- is converted to pyroglutamic acid at pH 2.0, with a halflife of about 24 hours for the conversion.

Normally Asp- would be expected to elute last from an anion-exchange column run in the HILIC mode, reflecting both its charge and the hydrophilic character which exceeds that of Glu- and cysteic acid. A pH of 2.0 is low enough to substantially uncharge its functional group, permitting its elution in the same time frame as the other amino acids (unless the wrong salt is used, cf. FIG. 10). At pH 4.0, Asp does indeed elute appreciably later than the other amino acids unless the electrostatic effects are antagonized by addition of more salt to the mobile phase (FIG. 12). Pyroglutamic acid also has a net negative charge at pH 4.0 and its retention time also decreases significantly (9′ to 6′) when the salt concentration increases from 10 to 20 mM.

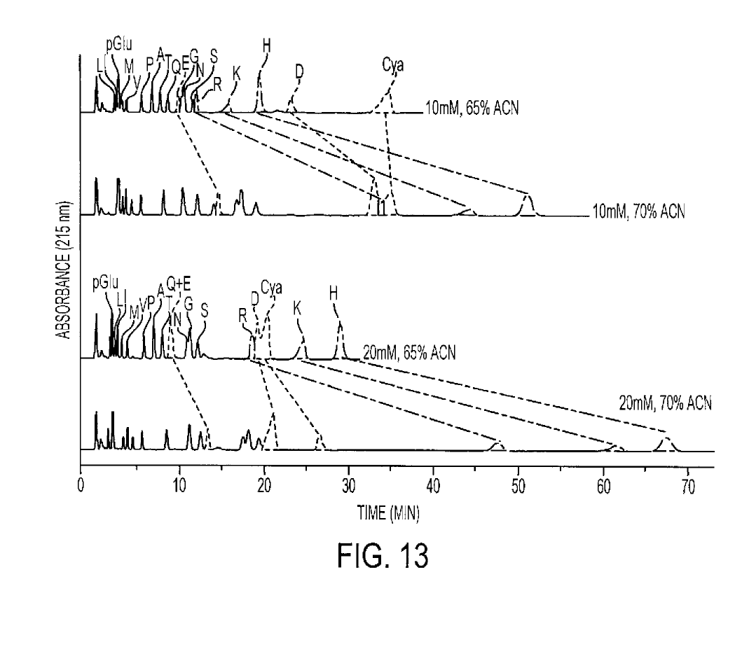

When the ACN concentration in the mobile phase is increased from 65 to 70%, retention times of all amino acids increase with the increase in the magnitude of hydrophilic interaction (FIG. 13). The most pronounced effect is the increase in retention of the basic amino acids—the most hydrophilic of all—to the point that they no longer elute in the same time frame as the other amino acids even when they and the polar column material bear the same charge. The retention of cysteic acid is notably unaffected by this change in ACN concentration. Cysteic acid, used here as a standard in place of cysteine, appears to be one of the more hydrophobic amino acids whose retention here is due almost entirely to electrostatic attraction. It retains its negative charge even at pH 2.0 (pK₁˜1.3). At pH 4.0 the disparity in charge relative to Glu and Asp is appreciably less.

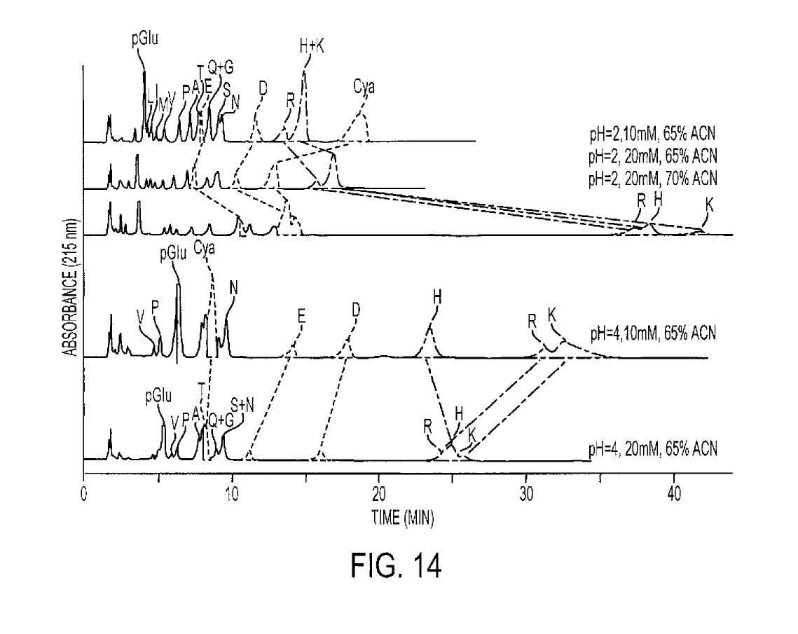

It is instructive to run amino acids on a PolyHYDROXYETHYL A™ column under the same conditions. The covalently-attached coating is poly(2-hydroxyethyl aspartamide), a neutral polypeptide with free N- and C-termini. Thus, the coating potentially has some positive and negative charge, albeit at a much lower level than does a regular ion-exchange material. At a pH of 4.4 these charges are in balance and the coating is in effect a neutral zwitterion. Above that pH, the net charge is negative; below, positive. At pH 2.0, where the coating has a modest overall positive charge, an increase in the salt concentration in the mobile phase increases retention of basic amino acids and decreases retention of acidic amino acids (FIG. 14), as with the PolyWAX LP™ column. However, at pH 4.0, the coating is near neutrality and an increasing level of salt decreases retention of both basic and acidic amino acids (with the exception of a modest increase in the retention of His). Again, increasing the level of ACN increases hydrophilic interaction and retention for all amino acids, the basic ones in particular (FIG. 14).

The use of nonvolatile salts in HILIC mobile phases is merely a matter of convenience, since salts such as triethylamine phosphate and sodium methylphosphonate permit the use of absorbance detection at low wavelengths and buffer at convenient pH ranges. As with any other essentially neutral stationary phase, PolyHYDROXYETHYL ATM can be used with volatile salts or unbuffered acids as electrolyte additives or even with no additive if a solute is not an electrolyte.

### III. ERLIC of Nucleotides and Nucleic Acids

A. HILIC vs. ERLIC

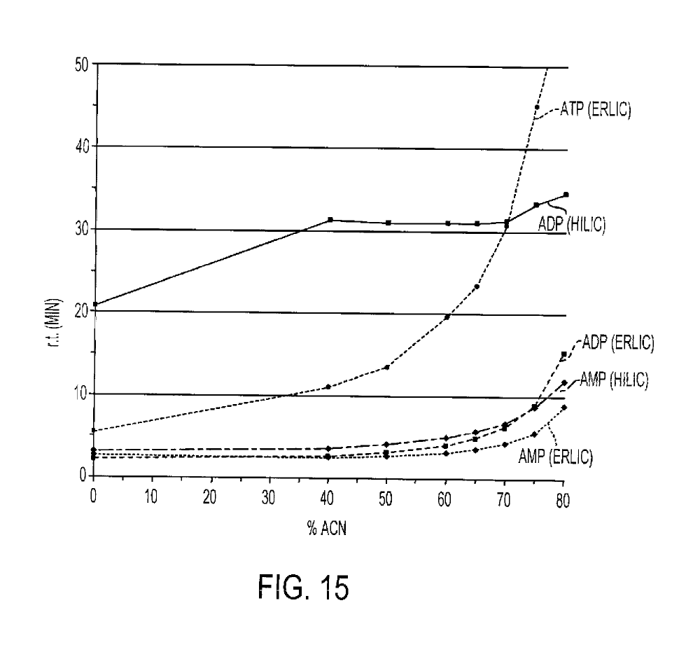

Nucleotides and nucleic acids possess negatively-charged phosphate groups. Therefore, ERLIC of these compounds was performed with a cation-exchange column. FIG. 15 compares the results with HILIC of these compounds on a column of a neutral material, PolyHYDROXYETHYL A™. At low concentrations of ACN where hydrophilic interactions are negligible, ADP elutes earlier than AMP from the cation-exchange column due to its greater electrostatic repulsion. At higher levels of ACN, where hydrophilic interactions with the phosphate groups become significant, their elution order is reversed. One would expect ATP to elute earlier than ADP at low levels of ACN. Its greater retention, seemingly anomalous, is discussed later. With the neutral column, the difference in retention between AMP, ADP and ATP is much greater, due to the lack of electrostatic repulsion and the great polarity of phosphate groups. This is especially the case here with ADP (ATP did not elute from the neutral column in a reasonable time under these conditions).

B. Effect of pH in ERLIC

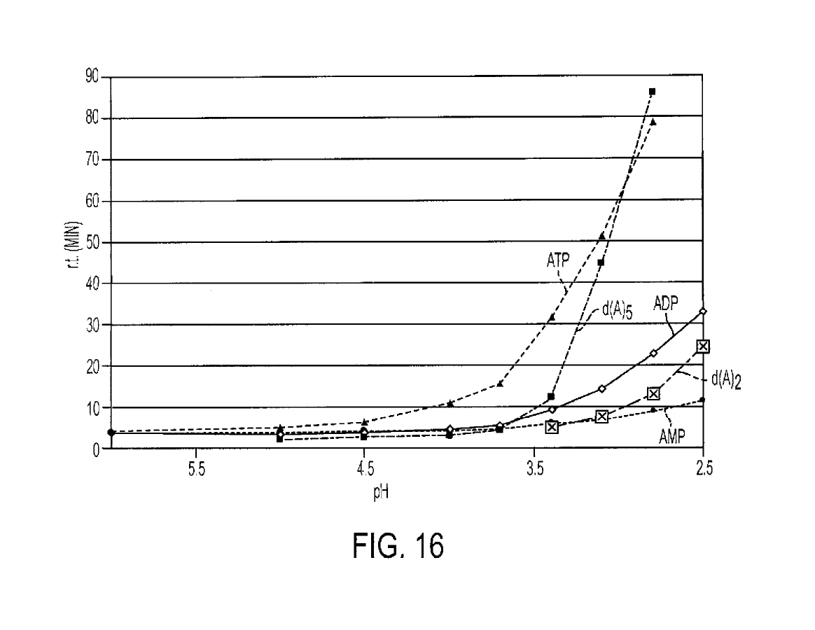

At pH 6, where phosphate groups are beginning to acquire their second negative charge, electrostatic repulsion is so great that no nucleotide or oligonucleotide is retained (FIG. 16). Retention increases with decreasing pH, particularly below pH 3.4 where the phosphate groups begin to lose their single negative charge. This effect is especially pronounced with the solutes containing the most phosphates, ATP and d(A)₅. With the less-phosphorylated solutes, it is difficult to separate the effect of decreasing negative charge on the phosphate groups from that of the increasing positive charge (+1→+2) on the adenine rings (pKa@3.6-4.0, depending on the nucleotide).

C. Effect of Salt Concentration and Identity on Selectivity

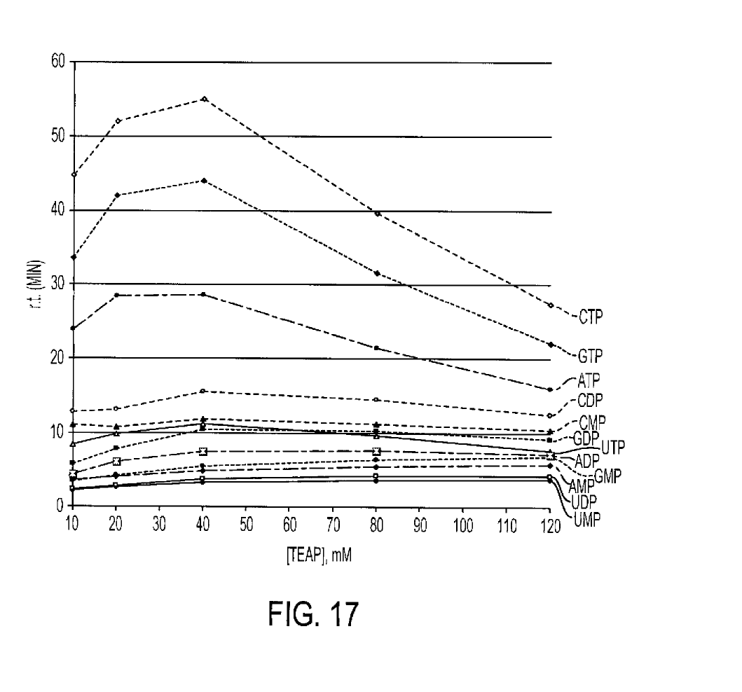

FIG. 17 shows that the effect of the base on retention in ERLIC is U˜T<A<G<C. At the ACN level used here, phosphorylation promotes retention in every case. Increasing salt increases the retention of UMP, AMP and GMP (but not CMP), indicating that electrostatic repulsion is a significant factor in their retention throughout the range. By contrast, the retention of di- and triphosphonucleotides increases to a maximum at 40 mM salt and falls off thereafter. A possible interpretation is that 40 mM salt is sufficient to shield most of the repulsive effects, while higher concentrations shield the electrostatic attraction of the positively-charged base for the stationary phase. The mechanism of this effect is discussed later.

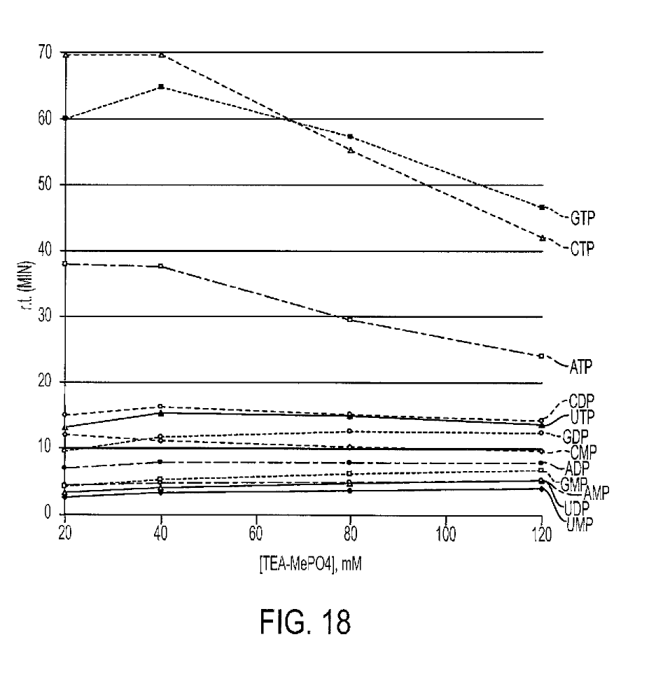

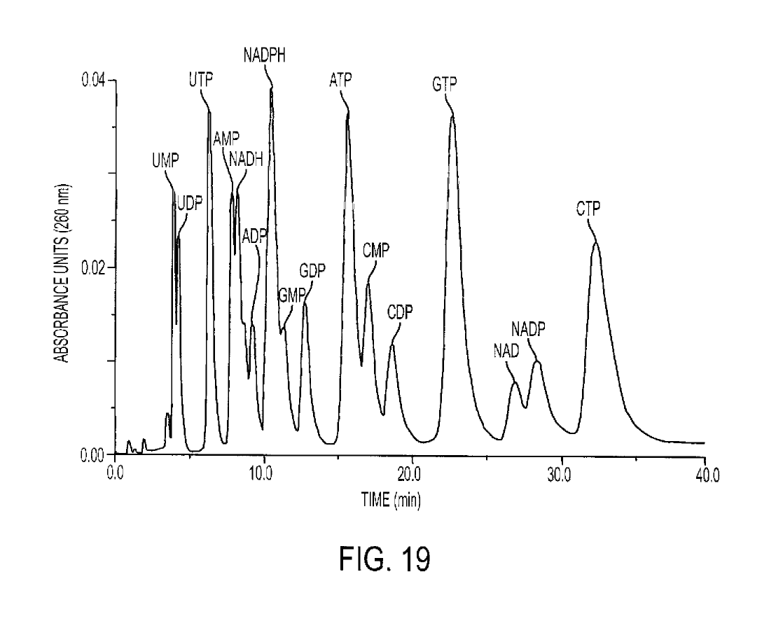

FIG. 18 displays the results obtained with TEA-MePO₄ substituted for TEAP. There is an increase in sensitivity to the number of phosphate groups at the expense of sensitivity to the base involved. Thus, there is a significant increase in the retention of the triphosphonucleotides relative to the retention of the mono- and diphosphonucleotides; The mechanism of this change is addressed later. TEAP is the better of the two salts with regard to isocratic elution of all the common nucleotides in the same time frame (FIG. 19).

A. Some Applications for ERLIC

Using general-purpose isocratic conditions, ERLIC is capable of obtaining separations of electrolyte mixtures that normally would require gradients. The only other mode of chromatography that routinely performs separations isocratically under standardized running conditions is Size Exclusion Chromatography (SEC). The resolution of SEC is limited to the number of peaks that can fit into the range between Vo and Vt. No such limitation pertains to ERLIC; the elution window can be widened merely by increasing the amount of organic solvent in the mobile phase. This affects the selectivity, since polarity effects then assume greater importance compared to electrostatic effects, so the utility of this approach should be assessed on a case-by-case basis. Nonetheless, certain general-purpose running conditions seem to suffice for a wide range of solutes. This should simplify methods development considerably. Not all mixtures will lend themselves to such treatment. No chromatographic method will afford complete separation of all the components in a protein digest that contains over 50 peptides, for example. However, complete separation is not necessary in every case. For example, if a mass spectrometer is used as the detector, it is only necessary to reduce the number of peptides coeluting to an extent that they do not interfere with each others' ionization. In that case, one could use an automatic sample injector to analyze a large number of samples rapidly, injecting each sample after the ERLIC window of elution of the preceding sample. The use of isocratic elution would simplify the equipment needed. ERLIC could be useful for separations performed on a silica wafer or chip, in which many samples might be analyzed simultaneously in numerous channels on a minute scale. Flow rates for such applications would be on the order of nanoliters per minute. It would greatly simplify the equipment needed for such separations as well if they could be performed isocratically. Finally, the advent of bottom-up or shotgun proteomics has increased the demand for alternative ways to fractionate complex mixtures of peptides in multidimensional approaches. ERLIC is a promising complement to current modes of chromatography.

Detection with mass spectrometry or an evaporative light scattering detector will require the development of ERLIC mobile phases based on volatile salts. The pronounced effect of counterions on retention in ERLIC complicates any attempt to substitute volatile salts for nonvolatile salts in the mobile phase. Maintaining a particular combination of selectivity may require careful matching of polarity and steric hindrance of the ions. Ammonium acetate or ammonium propionate may prove to be a suitable substitute for sodium methylphosphonate as long as a pH above 3 is satisfactory for an application, while triethylamine formate may be a satisfactory substitute for triethylamine phosphate.

B. Mechanism of Selectivity Effects

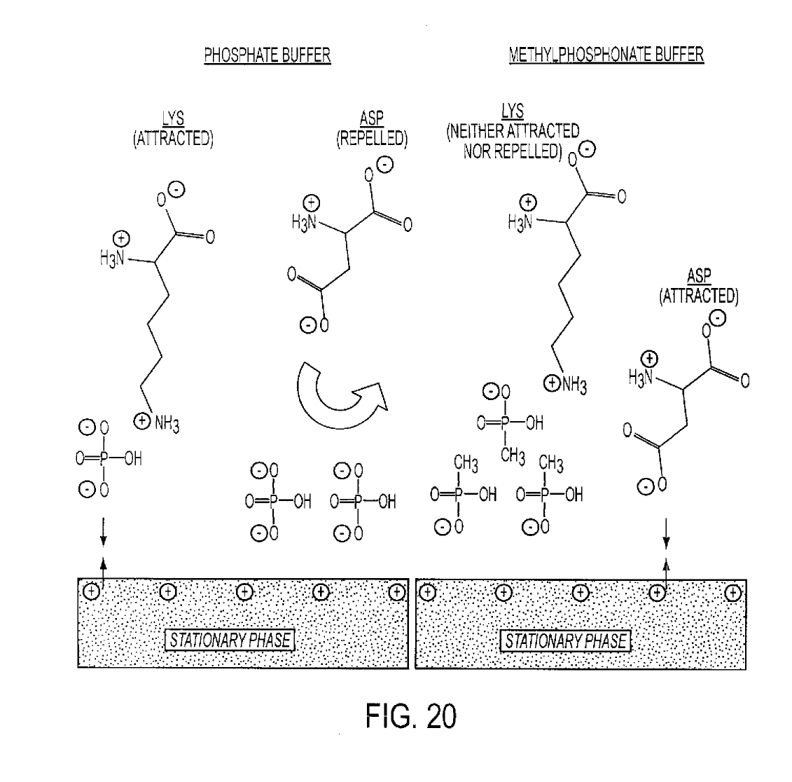

The selection of the salt in ERLIC can have a dramatic effect on selectivity. This can be accounted for if one assumes that these solutes, while small, can nonetheless be oriented in a rigid manner during their migration through the HPLC column. This has already been demonstrated with disaccharides in HILIC, for example [11]. FIG. 20 is a schematic of the orientation of amino acids in ERLIC. With phosphate as the counterion, its potential second negative charge provides a means for the attraction of basic amino acids to the surface. The potential for inducing a second accessible negative charge in methylphosphonate ion is significantly less. This would account for the observation that basic amino acids and peptides are better-retained in ERLIC with phosphate than with methylphosphonate as the counterion. By contrast, acidic amino acids would be repelled by the negatively-charged layer of phosphate ions on the surface of the stationary phase and hence elute rapidly with TEAP buffers unless the concentration of TEAP is too low to afford complete coverage of the surface. FIG. 12 suggests that that is the case below 20 mM TEAP.

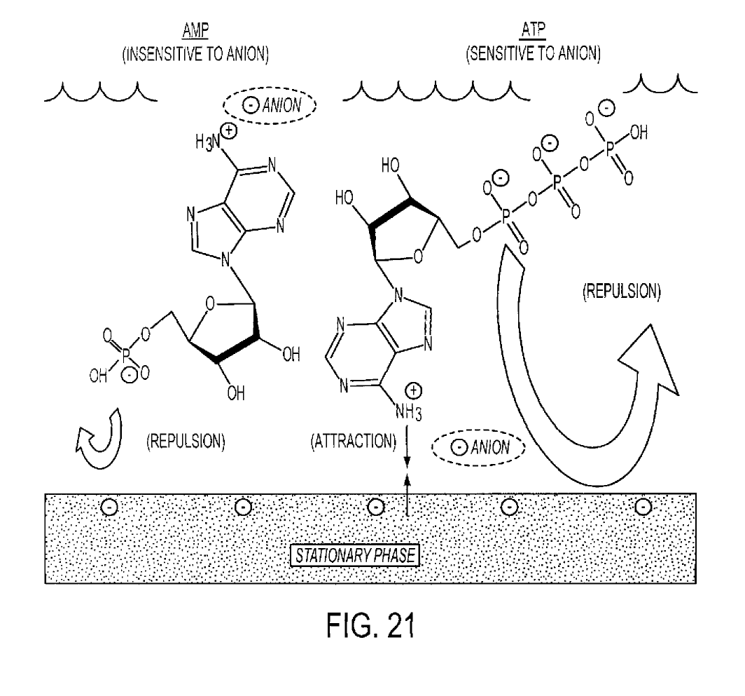

Orientation effects with nucleotides appear to be more complicated. FIG. 21 is a schematic contrasting the orientation of AMP and ATP. The phosphate group of AMP, being quite hydrophilic, is oriented toward the stationary phase but is repelled by it. Increasing salt concentrations suppress the repulsion and increase the retention of AMP. The nature of the counterion associated with the positively-charged base has little influence on retention. With ATP, the repulsion of the three phosphate groups by the stationary phase is so strong that they are oriented away from it. With the base now facing the stationary phase, retention is strongly influenced by the nature of the counterion, as in FIG. 17. This inverted orientation also accounts for the retention of ATP on a cation-exchange column in the absence of ACN and hydrophilic interaction (FIG. 15). ADP, not having the base oriented so rigidly toward the stationary phase, elutes in the void volume under these conditions. With sufficient ACN, the hydrophilicity of phosphate groups is such that they confer net retention on a molecule whatever their orientation.

A similar rationale can be applied to the orientation of peptides in ERLIC. Basic residues are likely oriented away from the stationary phase, even if they augment the net retention of the peptide. This would enhance selectivity for neutral and acidic residues. Thus, ERLIC should be able to afford separations that would be difficult to obtain in other modes of chromatography, including HILIC. A cautionary note is that ERLIC will not work if a solute does not have an orientation or a domain that is not repelled by the stationary phase.

It will be understood that while the invention has been described in conjunction with specific embodiments thereof, the foregoing description and examples are intended to illustrate, but not limit the scope of the invention. Other aspects, advantages and modifications will be apparent to those skilled in the art to which the invention pertains, and these aspects and modifications are within the scope of the invention and described and claimed herein.

### REFERENCES

1. A. J. Alpert, J. Chromatogr. 499 (1990) 177.
2. J. C. Linden and C. L. Lawhead, J. Chromatogr. 105 (1975) 125.
3. J. K. Palmer, Anal. Lett. 8 (1975) 215.
4. T. Wieland and H. Determann, J. Chromatogr. 28 (1967) 2.
5. J. Zhang and D. I. C. Wang, J. Chromatogr. B, 712 (1998) 73.
6. K. M. Swiderek, T. D. Lee, and J. E. Shively, Meth. Enzymol. 271 (1996) 68.
7. K. Furuya, K. M. Schegg, H. Wang, D. S. King, and D. A. Schooley, Proc. Natl. Acad. Sci. USA 92 (1995) 12323.
8. J. A. Boutin, A-P. Emould, G. Ferry, A. Genton, and A. J. Alpert, J. Chromatogr. 583 (1992) 137.
9. K. F. Faull, G. J. Feistner, K. Conklin, P. Roepstorff, and P. C. Andrews, Neuropeptides 32 (1998) 339.
10. P. Kulanthaivel et al., J. Biol. Chem. 279 (2004) 36250.
11. A. J. Alpert, M. Shukla, A. K. Shukla, L. R. Zieske, S. W. Yuen, M. A. J. Ferguson, A. Mehlert, M. Pauly, and R. Orlando, J. Chromatogr. A, 676 (1994) 191.
12. P. Jenö, P. E. Scherer, U. Manning-Krieg, and M. Horst, Anal. Biochem. 215 (1993) 292.
13. M. J. Schmerr, R. C. Cutlip, and A. Jenny, J. Chromatogr. A, 802 (1998) 135.
14. M. J. Schmerr and A. Alpert, in Prions and Mad Cow Disease (B. K. Nunnally and I. S. Krull, eds.), Marcel Dekker, 2004, pp. 359-377.
15. J. Carroll, I. M. Fearnley and J. E. Walker, Proc. Natl. Acad. Sci. USA 103 (2006) 16170.
16. M. A. Strege, Anal. Chem. 70 (1998) 2439.
17. M. A. Strege, Amer. Pharm. Rev. 2 (no. 3, 1999) 53.
18. S. Soltysik, D. A. Bedore, and C. R. Kensil, Ann. NY Acad. Sci. 690 (1993) 392.
19. Ph. Dallet, L. Labat, E. Kummer, and J. P. Dubost, J. Chromatogr. B, 742 (2000) 447.
20. M. McGrane, H. J. Keukens, M. O'Keeffe, J. A. van Rhijn and M. R. Smyth, EuroResidue IV (L. A. van Ginkel and A. Ruiter, eds.), 2000, pp. 765-770.
21. J. K. Troyer, K. K. Stephenson, and J. W. Fahey, J. Chromatogr. A, 919 (2001) 299.
22. Y.-J. Chen, D. R. Wing, G. R. Guile, R. A. Dwek, D. J. Harvey and S. Zamze, Eur. J. Biochem. 251 (1998) 691.
23. E. S. M. Ng, F. Yang, A. Kameyama, M. M. Palcic, O. Hindsgaul and D. C. Schreimer, Anal. Chem. 77 (2005) 6125.
24. G. Karlsson, A.-C. Hinz and S. Winge, J. Chromatogr. Sci. 42 (2004) 361.
25. S. D. Garbis, A. Melse-Boonstra, C. E. West and R. B. van Breemen, Anal. Chem. 73 (2001) 5358.
26. T. P. Moyer, J. R. Charlson, R. J. Enger, L. C. Dale, J. O. Ebbert, D. R. Schroeder and R. D. Hurt, Clin. Chem. 48 (2002) 1460.
27. B. Zywicki, G. Catchpole, J. Draper and O. Fiehn, Anal. Biochem. 336 (2005) 178.
28. T. Yoshida, Anal. Chem. 69 (1997) 3038
29. T. Yoshida, J. Chromatogr. A, 808 (1998) 105.
30. T. Yoshida, J. Biochem. Biophys. Methods 60 (2004) 265.
31. P. Hemström and K. Irgum, J. Sep. Sci. 29 (2006) 1784.
32. H. Lindner, B. Sarg, and W. Helliger, J. Chromatogr. A, 782 (1997) 55.
33. H. Lindner, B. Sarg, B. Hoertnagl, and W. Helliger, J. Biol. Chem. 273 (1998) 13324.
34. C. A. Mizzen, A. J. Alpert, L. Levesque, T. P. A. Kruck and D. R. McLachlan, J. Chromatogr. B, 744 (2000) 33.
35. B. Sarg, A. Green, P. Soderkvist, W. Helliger, I. Rundquist and H. H. Lindner, FEBS J. 272 (2005) 3673.
36. B. Sarg, W. Helliger, H. Talasz, B. Forg and H. H. Lindner, J. Biol. Chem. 281 (2006) 6573.
37. J. Pesavento, C. A. Mizzen and N. L. Kelleher, Anal. Chem. 78 (2006) 4271.
38. C. T. Mant, L. H. Kondejewski, P. J. Cachia, O. D. Monera and R. S. Hodges, Meth. Enzymol. 289 (1997) 462-467.
39. C. T. Mant, J. R. Litowski, and R. S. Hodges, J. Chromatogr. A, 816 (1998) 65.
40. C. T. Mant, L. H. Kondejewski, and R. S. Hodges, J. Chromatogr. A, 816 (1998) 79.
41. A. J. Oosterkamp, E. Gelpi, and J. Abian, J. Mass Spectrom. 33 (1998) 976.
42. Y.-M. Zhang, M. W. Frank, K. G. Virga, R. E. Lee, C. O. Rock and S. Jackowski, J. Biol. Chem. 279 (2004) 50969.
43. H. D. Crone, J. Chromatogr. 107 (1975) 25.
44. A. Trifiro, G. Saccani, S. Gherardi, E. Vicini, E. Spotti, M. P. Previdi, M. Ndagijimana, S. Cavalli, and C. Reschiotto, J. Chromatogr. A, 770 (1997) 243.
45. L. Roden, M. Roden, H. Yu, J. Jin, and J. Greenshields, J. Chromatogr. 638 (1993) 29.
46. A. J. Alpert and P. C. Andrews, J. Chromatogr. 443 (1988) 85.
47. E. Bosch, P. Bou, H. Allemann, and M. Rosés, Anal. Chem. 68 (1996) 3651.
48. A. J. Alpert and F. E. Regnier, J. Chromatogr. 185 (1979) 375.
49. A. J. Alpert, submitted for publication.
50. A. J. Alpert, in Column Handbookfor Size Exclusion Chromatography (C.-S. Wu, ed.), Academic Press, 1999, pp. 249-266 [FIG. 8.7].
51. T. J. Higley and T. Yoshida, LCGC Application Notebook, June 2003, 20-21
52. Y. Guo and S. Gaiki, J. Chromatogr. A, 1074 (2005) 71.
53. Table 8.8, Lange's Handbook of Chemistry, 15^{th} ed. (John A. Dean, ed.), McGraw-Hill, Inc., 1999.
54. V. I. Teichberg, D. Aberdam, U. Erez and E. Pinelli, J. Biol. Chem. 263 (1988) 14086.
55. R. Nogueira, M. Lammerhofer and W. Lindner, J. Chromatogr. A, 1089 (2005) 158.
56. S.-M. Liu, L. Xu, C.-T. Wu and Y.-Q. Feng, Talanta 64 (2004) 929.
57. W. Jiang, G. Fischer, Y. Girmay and K. Irgum, J. Chromatogr. A, 1127 (2006) 82.

I claim:

1. A method of performing electrostatic repulsion-hydrophilic interaction chromatography on a compound selected from the group consisting of proteins, peptides, amino acids, and charged derivatives thereof, the method comprising:
providing a column having an anion-exchange material at a pH of less than about 4; and
eluting the compound using a mobile phase comprising an amount of organic solvent sufficient to substantially balance the electrostatic repulsion of the stationary phase with hydrophilic interaction.

2. The method of claim 1, wherein the compound is eluted isocratically.

3. The method of claim 1, wherein the compound is eluted using a gradient of salt concentration, pH, polarity, or a combination thereof.

4. The method of claim 1, wherein the compound is a charged derivative of a molecule selected from the group consisting of proteins, peptides, and amino acids.

5. The method of claim 4, wherein the charged derivative comprises a phosphate group or a sulfate group.

6. The method of claim 1, wherein the polarity of the mobile phase is increased or decreased by adjusting the concentration in the mobile phase of a solvent selected from the group consisting of water, acetonitrile, methanol, ethanol, propanol, and other solvents suitable for HILIC.

7. The method of claim 1, wherein the net charge of the stationary phase is increased or decreased by altering the pH of the mobile phase.

8. The method of claim 3 wherein the salt is selected from the group consisting of triethylamine phosphate, triethylamine methylphosphonate, sodium methylphosphonate, and other salts compatible with HILIC mobile phases.

9. A method of performing electrostatic repulsion-hydrophilic interaction chromatography on a compound selected from the group consisting of nucleic acids, nucleotides, and charged derivatives thereof, the method comprising:
providing a column having a cation-exchange material at a pH of less than about 3.4; and
eluting the compound using a mobile phase comprising organic solvent sufficient to substantially balance the electrostatic repulsion of the stationary phase with hydrophilic interaction.

10. The method of claim 9, wherein the compound is eluted isocratically.

11. The method of claim 9, wherein the compound is eluted using a gradient of salt concentration, pH, polarity, decreasing organic solvent content, or a combination thereof.

12. The method of claim 9, wherein the compound is a charged derivative of a molecule selected from the group consisting of nucleic acids and nucleotides.

13. The method of claim 9, wherein the polarity of the mobile phase is increased or decreased by adjusting the concentration in the mobile phase of a solvent selected from the group consisting of water, acetonitrile, methanol, ethanol, propanol, and other solvents suitable for HILIC.

14. The method of claim 9, wherein the net charge of the stationary phase is increased or decreased by altering the pH of the mobile phase.

15. The method of claim 11 wherein the salt is selected from the group consisting of triethylamine phosphate, triethylamine methylphosphonate, sodium methylphosphonate, and other salts compatible with HILIC mobile phases.

16. A method of performing electrostatic repulsion-hydrophilic interaction chromatography on a phosphopeptide compound, the method comprising:
providing a column having an anion-exchange material at a pH of less than about 4; and
eluting the compound using a mobile phase comprising an amount of organic solvent sufficient to substantially balance the electrostatic repulsion of the stationary phase with hydrophilic interaction.

17. The method of claim 16, wherein the compound is eluted isocratically.

18. The method of claim 16, wherein the compound is eluted using a gradient of salt concentration, pH, polarity, or a combination thereof.

19. The method of claim 16, wherein the polarity of the mobile phase is increased or decreased by adjusting the concentration in the mobile phase of a solvent selected from the group consisting of water, acetonitrile, methanol, ethanol, propanol, and other solvents suitable for HILIC.

20. The method of claim 18 wherein the salt is selected from the group consisting of triethylamine phosphate, triethylamine methylphosphonate, sodium methylphosphonate, and other salts compatible with HILIC mobile phases.
